(19) United States
(12) Patent Application Publication
(10) Pub. No.: US 2003/0092688 A1
(43) Pub. Date: May 15, 2003

(54) Non-combustible water-dispersible vitamin compositions

(76) Inventor: Michel Andre Crepeau, Pueblo West, CO (US)

Correspondence Address: CONNOLLY BOVE LODGE & HUTZ, LLP, 1220 N MARKET STREET, P O BOX 2207, WILMINGTON, DE, 19899

(21) Appl. No.: 09/921,947

(22) Filed: Aug. 3, 2001

(51) Int. Cl. A61K 31/355; A61K 31/07

(52) U.S. Cl. 514/168; 514/458; 514/725

(57) ABSTRACT

A water-dispersible and substantially non-combustible liquid vitamin composition comprising one or more of Vitamins A, D3 and E, or a precursor thereof, and an emulsifier, an alkyl lactate and water.

The present invention relates to new water-dispersible compositions to provide one or more of vitamins E, A and D₃ to an animal.

The present invention provides a water-dispersible and substantially non-combustible liquid vitamin composition comprising:

a) from 10% to 60% by weight of a vitamin component selected from the group consisting of:

(i) one or more precursors of Vitamin A;

(ii) one or more precursors of Vitamin E;

(iii) a mixture of one or more precursors of Vitamin A and one or more precursors of Vitamin E;

(iv) a mixture of one or more precursors of Vitamin A and Vitamin D3;

(v) a mixture of one or more precursors of Vitamin E and Vitamin D3; and

(vi) a mixture of one or more precursors of Vitamin A, one or more precursors of Vitamin E, and Vitamin D3;

b) from 2% to 15% by weight of a C4 to C6 alkyl lactate;

c) from 20% to 50% by weight of one or more veterinarily acceptable emulsifiers;

d) from 1% to 15% by weight of water; and

e) from 2% to 10% of an oil;

wherein the flashpoint of the composition is about 200° F. or greater.

The present invention also provides a water-dispersible and substantially non-combustible liquid vitamin composition comprising:

a) from 10% to 60% by weight of a vitamin component selected from the group consisting of:

(i) one or more precursors of Vitamin A;

(ii) one or more precursors of Vitamin E;

(iii) a mixture of one or more precursors of Vitamin A and one or more precursors of Vitamin E;

(iv) a mixture of one or more precursors of Vitamin A and Vitamin D3;

(v) a mixture of one or more precursors of Vitamin E and Vitamin D3; and

(vi) a mixture of one or more precursors of Vitamin A, one or more precursors of Vitamin E, and Vitamin D3;

b) from 3% to 15% by weight of a C1 to C3 alkyl lactate;

c) from 20% to 50% by weight of one or more veterinarily acceptable emulsifiers;

d) from 3% to 15% by weight of water; and

e) from 2 to 10% of an oil;

wherein the flashpoint of the composition is about 200° F. or greater.

The present invention also provides a water-dispersible and substantially non-combustible liquid vitamin composition comprising:

a) from 1% to 6% by weight of Vitamin D3;

b) from 2% to 15% by weight of a C4 to C6 alkyl lactate;

c) from 20% to 50% by weight of one or more veterinarily acceptable emulsifiers;

d) from 1% to 15% by weight of water; and

e) from 5% to 30% by weight of an oil;

wherein the flashpoint of the composition is about 200° F. or greater.

The present invention also provides a water-dispersible and substantially non-combustible liquid vitamin composition comprising:

a) from 1% to 6% by weight of Vitamin D3:

b) from 3% to 15% by weight of a C1 to C3 alkyl lactate;

c) from 20% to 50% by weight of one or more veterinarily acceptable emulsifiers;

d) from 1% to 15% by weight of water; and

e) from 5% to 30% by weight of an oil;

wherein the flashpoint of the composition is about 200° F. or greater.

It should be understood that in the present specification and claims the term “% ” means percent by weight unless expressly noted otherwise.

In certain embodiments of the present invention, the liquid vitamin compositions consist essentially of the ingredients listed in the foregoing descriptions of the compositions.

The vitamins are suitably provided as the oily derivative of the vitamin, such as the lower alkyl esters of the vitamin as a solution or suspension in an oil (e.g., a veterinarily acceptable oil). By the term lower alkyl is generally meant C1-C6 alkyl which is optionally substituted by one or more halogens. For example Vitamin A may be provided as retinyl propionate. Vitamin E may be provided as, e.g, (dl) alpha tocopheryl acetate. Suppliers of the vitamins or precursors thereof include Hoffman LaRoche Corporation, and Aventis Animal Nutrition. Such products are generally known to those skilled in the art.

The oil is, e.g., a fatty acid oil, such as a vegetable oil, as needed to provide the compositions. Such oils are acceptable as food additives and known to the person skilled in the art. Such oils include soybean oil, corn oil, canola oil, peanut oil and the like. In the case of vitamin E, or its precursor dl-alpha tocopheryl acetate, the oil may be composed of or include a manufacturing by-product.

Alkyl lactates that may be used according to the invention include methyl lactate, ethyl lactate, n-propyl lactate, iso-propyl lactate, n-butyl lactate, iso-butyl lactate, sec-butyl lactate, tert-butyl lactate, n-pentyl lactate, n-hexyl lactate and other isomeric forms thereof. The alcohol portion of the ester may be optionally substituted by one or more halogens. Ethyl lactate and butyl lactate are examples of preferred alkyl lactates that are used in the invention. All enantiomeric and diasteromeric forms of lactate esters are embraced by the present invention. Lactate esters of L(+) lactic acid are generally preferred.

In certain embodiments of the present invention the amount of water in the composition is from 1 to 15%, preferably from 1 to 10% by weight, more preferably from 1 to 5% by weight. In another embodiment of the present invention, (specifically the C1-C3 alkyl lactate embodiment) the amount of water in the composition is from 3 to 15%, preferably from 3 to 10%, most preferably from 3 to 6% by weight.

In another aspect of the invention, the compositions of the invention may further comprise from 1% to 15% by weight of a veterinarily acceptable stabilizer. The stabilizer enhances vitamin stability and keeps the composition as a flowable liquid for an extended period of time, generally from 1 to 6 months. The stabilizer may also function as an anti-gelling agent and/or as an antifreeze. Suitable stabilizers include sorbitol, propane 1,2 diol (also known as propylene glycol) and glycerine. The preferred agents are sorbitol and propylene glycol.

When the composition comprises a C4-C6 alkyl lactate, the amount of alkyl lactate may be 4%-7% in certain preferred embodiments of the present invention. When the composition comprises a C1-C3 alkyl lactate then the amount of alkyl lactate may be 3%-12% in certain preferred embodiments of the present invention.

Another aspect of the present invention is the absence of flammable alcohols in the composition. Generally such flammable alcohols are monohydroxy alcohols, particularly C1-C6 alcohols such as methanol, ethanol, n-propanol, isopropanol, n-butanol, isobutanol, sec-butanol and tertiary butanol. By the term flammable is generally meant a substance having a flashpoint of 100° F. or less. Unless otherwise specified, the procedure to determine flashpoints is known as the ISO 2719, closed cup method.

The compositions of the invention may also comprise from 0.5% to 6% by weight, preferably 3%, of an antioxidant which antioxidant may be ethoxyquin, BHT, or BHA.

When the composition comprises a C4-C6 alkyl lactate, the ratio of alkyl lactate to water may be from 1:5 to 10:1. When the composition comprises a C1-C3 alkyl lactate then the alkyl lactate:water ratio may be from 1:5 to 4:1.

The emulsifiers according to the present invention may be any type of emulsifiers, and are preferably non-ionic surfactants. Examples of non-ionic surfactants include polyethylene glycol esters and ethoxylated sorbitan fatty acid esters. Examples of these groups of surfactants include Polysorbate 80, Polysorbate 80K, PEG 400, Alkamuls® PSMO-20, Alkamuls® 400-MO, T-MAZ 80K, MAPEG® 400Mo and the like and are generally known to those skilled in the art.

The compositions of the present invention may have viscosities that are from 1000 cP to 10000 cP at 0° C. and from 100 cP to 2000 cP at 15° C. It is understood that viscosities are measured by the Brookfield method known to those of skill in the art and specifically described in Example 7 of this application.

Another aspect of the present invention is that the composition may be quickly dispersed in water. That is the composition adequately disperses into water within 2 minutes, preferably within 20 seconds, when added at a ratio of composition to water of from 1 g/kg to 50 g/kg, preferably from 3 to 10 g/kg. By the term “adequately disperses” is meant that the composition disperses into water and forms a finely dispersed emulsion (e.g., under the conditions specified in Example 6).

The composition may also comprise a fungicide. Any suitable fungicide acceptable in veterinary medicine may be used and in particular potassium sorbate is preferred. When a fungicide is used, it is present in the composition in trace amounts, for example, from about 0.05% to 0.3% by weight of the composition.

The invention also includes a packaged product comprising the composition of the invention which may be shipped and stored in the United States of America without a restriction due to flammability or combustibility as understood within the Code of Federal Regulations, Materials Regulations and Procedures, Chapter 1, Subpart C, Section 173.120. Such a product may be shipped at much lower cost per unit weight of vitamin and may be stored without fear of explosion, flammability or combustibility.

Specific compositions for delivery to animals contemplated by the invention include those of Table 1. Stock solutions or suspensions of vitamins and vitamin precursors are generally supplied at specific International potency units per gram (IU/g). For example, the precursor to Vitamin A used in the formulations of Table 1 is retinyl propionate which is used as a 79% by weight solution of retinyl propionate dissolved in canola oil and stabilized with 1% by weight ethoxyquin. This means that the retinyl propionate/canola oil solution contains about 2,200,000 IU/g of Vitamin A (pure all trans retinyl propionate has a theoretical potency of 2,780,000 IU/g). The Vitamin D3 composition used in Table 1 is an oily concentrate prepared from Vitamin D3 resin and contains about 10% by weight Vitamin D3 in vegetable oil. The Vitamin D3/vegetable oil concentrate is stabilized with BHA or BHT and has about 4,000,000 IU/g of Vitamin D3 (pure cholecalciferol, also known as Vitamin D3, has a theoretical potency of 40,000,000 IU/g). The Vitamin E precursor used in Table 1 is 94.5% by weight pure dl-alpha tocopheryl acetate in oil (945 IU/g).

TABLE 1

| | | | | % weight of | |
|---|---|---|---|---|---|
| Product | | | Vitamin | Vitamin or | % oil |
| Name | Vitamin | Precursor | Potency (IU/g) | precursor | (by weight) |
| A400 | A | Retinyl propionate | 400,000 | 15.7 | 3.9 |
| A500 | A | Retinyl propionate | 500,000 | 19.7 | 4.8 |
| A1000 | A | Retinyl propionate | 1,000,000 | 39.4 | 9.6 |
| AD3 | A | Retinyl propionate | 500,000 | 19.7 | 4.8 |
| 500/100 | D3 | none | 100,000 | 0.26 | 2.34 |
| AD3E 400- | A | Retinyl propionate | 400,000 | 15.7 | 3.9 |
| 100-100 | | | | | |
| | D3 | none | 100,000 | 0.26 | 2.34 |
| | E | dl alpha tocopheryl | 100 | 10.3 | 0.6 |
| | | acetate | | | |
| E50 | E | dl alpha tocopheryl | 500 | 51.5 | 3.0 |
| | | acetate | | | |
| E40 | E | dl alpha tocopheryl | 400 | 41.2 | 2.4 |
| | | acetate | | | |
| D3 500 | D3 | none | 500,000 | 1.3 | 11.8 |
| 03 1000 | D3 | none | 1,000,000 | 2.6 | 23.6 |

When Vitamin D3 is present in a composition along with precursors of Vitamin A, the weight ratio of precursors of Vitamin A to Vitamin D3 may be from 100:1 to 40:1.

The concentration of each vitamin in the compositions may be varied to satisfy the specific requirements of the product desired. Generally, Vitamin A is present in an amount of from 300,000 to 1,200,000 IU/g, preferably from 400,000 to 1,000,000 IU/g. Vitamin D3 is generally present in an amount of from 50,000 to 2,000,000 IU/g, preferably from 100,000 to 1,000,000 IU/g. Vitamin E is generally present in an amount of from 50 to 500 IU/g, preferably from 100 to 500 IU/g.

### EXAMPLE 1

Table 2 Compositions. For each composition, an organic phase was produced as follows: Retinyl propionate (79% by weight in canola oil), Polysorbate 80, PEG 400 MO, Ethoxyquin and Alkyl Lactate in the amounts specified in Table 2 were mixed together until homogeneous. In a separate aqueous phase, Potassium Sorbate was added to distilled water and agitated until dissolved. Propylene Glycol was added to the aqueous phase and mixed until homogeneous. The aqueous phase was then added to the organic phase under agitation and mixed until homogeneous (generally two hours).

Compositions 1,2,3, and 9 are comparative examples.

TABLE 2

| Composition | | | | | | | | |
|---|---|---|---|---|---|---|---|---|
| No. | 1 | 2 | 3 | 4 | 5 | 6 | 7 | 8 |
| Water | 0 | 1 | 2 | 3 | 4 | 5 | 10 | 10 |
| Potassium | 0.1 | 0.1 | 0.1 | 0.1 | 0.1 | 0.1 | 0.1 | 0.1 |
| Sorbate | | | | | | | | |
| Propylene | 15 | 15 | 15 | 15 | 15 | 15 | 15 | 15 |
| Glycol | | | | | | | | |
| Methyl Lactate | 10 | 10 | 10 | 10 | 10 | 10 | 4 | 10 |
| Polysorbate 80 | 23.7 | 23.2 | 22.7 | 22.2 | 21.7 | 21.2 | 21.7 | 18.7 |
| PEG 400 | 23.7 | 23.2 | 22.7 | 22.2 | 21.7 | 21.2 | 21.7 | 18.7 |
| Ethoxyquin | 3 | 3 | 3 | 3 | 3 | 3 | 3 | 3 |
| retinyl | 24.5 | 24.5 | 24.5 | 24.5 | 24.5 | 24.5 | 24.5 | 24 5 |
| propionate | | | | | | | | |
| Total | 100 | 100 | 100 | 100 | 100 | 100 | 100 | 100 |
| Water Alkyl | 0 | 0.1 | 0.2 | 0.3 | 0.4 | 0.5 | 2.5 | 1 |
| Lactate | | | | | | | | |
| Observed | <140 | 140 | 158 | >200 | >200 | >200 | >200 | >200 |
| FP (F) | | | | | | | | |
| Composition | 9 | 10 | 11 | 12 | 13 | 14 | 15 | 16 |
| No. | | | | | | | | |
| Water | 0 | 1 | 2 | 3 | 4 | 5 | 10 | 10 |
| Potassum | 0.1 | 0.1 | 0.1 | 0.1 | 0.1 | 0.1 | 0.1 | 0.1 |
| Sorbate | | | | | | | | |
| Propylene | 15 | 15 | 15 | 15 | 15 | 15 | 15 | 15 |
| Glycol | | | | | | | | |
| Butyl Lactate | 10 | 10 | 10 | 10 | 10 | 10 | 4 | 10 |
| Polysorbate 80 | 23.7 | 23.2 | 22.7 | 22.2 | 21.7 | 21.2 | 21.7 | 18.7 |
| PEG 400 | 23.7 | 23.2 | 22.7 | 22.2 | 21.7 | 21.2 | 21 7 | 18.7 |
| Ethoxyquin | 3 | 3 | 3 | 3 | 3 | 3 | 3 | 3 |
| retinyl | 24.5 | 24.5 | 24.5 | 24.5 | 24.5 | 24.5 | 24.5 | 24.5 |
| propionate | | | | | | | | |
| Total | 100 | 100 | 100 | 100 | 100 | 100 | 100 | 100 |
| Water/Alkyl | 0 | 0.1 | 0.2 | 0.3 | 0.4 | 0.5 | 25 | 1 |
| Lactate | | | | | | | | |
| Observed | 172 | >200 | >200 | >200 | >200 | >200 | >200 | >200 |
| FP (F) | | | | | | | | |

### EXAMPLE 2

The composition shown in Table 3 was produced by the method described in this example.

Retinyl propionate (79% in canola oil), Alkamuls PSMO-20, Alkamuls 400-MO, Ethoxyquin and Butyl Lactate were mixed together until homogeneous. In an aqueous phase, Potassium Sorbate was added to distilled water and agitated until dissolved. Propylene Glycol was added and mixed until homogeneous. The aqueous phase was added to the organic phase under agitation and mixed until homogeneous (generally two hours).

TABLE 3

| Ingredient | % weight | kg |
|---|---|---|
| Distilled Water | 10.00 | 18.1 |
| Potassium Sorbate | 0.10 | 0.2 |
| Propylene Glycol | 15.00 | 27.4 |
| Butyl Lactate | 4.00 | 7.3 |
| Alkamuls PSMO-20 | 21.71 | 3g.5 |
| Alkamuls 400-MO | 21.71 | 3g.5 |
| Ethoxyquin | 3.00 | 5.4 |
| Retinyl Propionate 79% in oil | 24.48 | 44.6 |
| Total | 100 | 136.26 |

The flashpoint was measured as >200° F.

### EXAMPLE 3

The composition shown in Table 4 was produced by the method described in this example. A precursor to Vitamin E (94.5% by weight dl alpha tocopheryl acetate in oil), Alkamuls PSMO-20, Alkamuls 400-MO, and butyl lactate were mixed together until homogeneous. In a separate aqueous phase, potassium sorbate was added to distilled water and agitated until dissolved. Propylene glycol was added to the aqueous phase and mixed until homogeneous. The aqueous phase was added to the organic phase under agitation and mixed until homogeneous (generally two hours).

TABLE 4

| Ingredient | % weight | kg |
|---|---|---|
| Distilled Water | 6.00 | 5.4 |
| Potassium Sorbate | 0.10 | 0.1 |
| Propylene Glycol | 3.00 | 2.7 |
| Butyl Lactate | 5.00 | 4.5 |
| Alkamuls PSMO-20 | 11.40 | 10.4 |
| Alkamuls 400-MO | 20.00 | 18.1 |
| dl alpha | 54.50 | 49.7 |
| tocopheryl | | |
| acetate | | |
| (94.5%) | | |
| Total | 100.00 | 91.0 |

The flashpoint was measured at >200° F.

### EXAMPLE 4

The composition shown in Table 5 was produced by the method described in this example. Vitamin D3 (10% by weight in vegetable oil), Polysorbate 80, PEG 400 MO, Ethoxyquin and Ethyl Lactate were mixed together until homogeneous. In a separate aqueous phase, Potassium Sorbate was added to water and agitated until dissolved. Propylene Glycol was added to the aqueous phase and mixed until homogeneous. The aqueous phase was added to the organic phase under agitation and mixed until homogeneous (generally two hours).

TABLE 5

| Ingredient | % weight | kg |
|---|---|---|
| Distilled Water | 11 .00 | 110.0 |
| Potassium Sorbate | 0.10 | 1.0 |
| Propylene Glycol | 11.00 | 110.0 |
| Ethyl Lactate | 5.00 | 50.0 |
| Polysorbate 80 | 28.38 | 283.8 |
| PEG 400-MO | 28.39 | 283.9 |
| Ethoxyquin | 3.00 | 30.0 |
| Vitamin D3 oil (10% in oil) | 13.13 | 131.1 |
| Total | 100 | 1000.0 |

The flashpoint was measured as >200° F.

### EXAMPLE 5

The composition shown in Table 6 was produced by the method described in this example. Retinyl Propionate (79% by weight in canola oil), Vitamin D3 (10% by weight in vegetable oil) and, (dl) alpha tocopheryl acetate (94.5% by weight in oil), Polysorbate 80, PEG 400 MO, Ethoxyquin and Ethyl Lactate were mixed together until homogeneous. In a separate aqueous phase, Potassium Sorbate was added to water and agitated until dissolved. Propylene Glycol was added and mixed until homogeneous. The aqueous phase was added to the organic phase under agitation and mixed until homogeneous (generally two hours).

TABLE 6

| Ingredient | % weight | kg |
|---|---|---|
| Distilled Water | 5.00 | 500.0 |
| Potassium Sorbate | 0.10 | 1.0 |
| Propylene Glycol | 15.00 | 150.0 |
| Ethyl Lactate | 7.00 | 70.0 |
| Polysorbate 80 | 15.00 | 150.0 |
| PEG 400-MO | 21.80 | 218.0 |
| Ethoxyquin | 3.00 | 30.0 |
| alpha dl tocopheryl acetate | 10.90 | 109.0 |
| (94.5%) | | |
| Retinyl Propionate 79% in | 19.58 | 195.8 |
| canola oil | | |
| Vitamin D3 (10% in oil) | 2.63 | 26.3 |
| Total | 100 | 1000.0 |

The flashpoint was measured as >200° F.

### EXAMPLE 6

Compositions 4-8 and 10-16 of Example 1 and the compositions of Examples 2, 3, 4 and 5 are tested for dispersion characteristics according to the following protocol.

About 5 drops (approx 0.15 g) of composition is added to 50 ml of water (room temperature which is from about 15° C. to about 25° C.) in a 100 ml flat-bottomed beaker and stirred with rod manually for 20 seconds. The resulting emulsion is examined and rated according to the following scale.

| | |
|---|---|
| 1. | No emulsion or dispersion; composition is separated from water |
| 2. | Partial emulsion/dispersion but large agglomerations of test |
| | product are observed |
| 3. | Almost complete emulsion/dispersion; small agglomerations |
| | are visible |
| 4. | Complete emulsion/dispersion; homogeneous liquid without any |
| | visible agglomerations |

All tested compositions of Examples 1-5 dispersed with a 4 rating.

### EXAMPLE 7

Viscosity is measured with a Brookfield DV-II+Viscometer (Brookfield Engineering Labs, Middleboro, Mass.) using RV spindle #3. The composition to be tested is placed in a 200 ml flat-bottomed sample jar and chilled at −20° C. for several hours. The viscometer functions in combination with a microcomputer supplied by Brookfield and uses a timed-stop program whereby a viscosity reading is taken every 30 seconds. Viscosity, torque and temperature are measured until the sample warms to about from 10° C. to 15° C.,

Representative viscosities are the following.

| | Viscosity | Viscosity | Viscosity |
|---|---|---|---|
| Example | at 0° C. (cP) | at 10° C. (cP) | at 15° C. (cP) |
| 1 (Composition 15) | 3200 | 780 | 500 |
| 5 | 4000 | 1050 | 750 |
| 3 | 7000 | 2250 | 1390 |

It should be understood that the preceeding is merely a detailed description of the embodiments of the invention and that numerous changes to the disclosed embodiments can be made in accordance with the disclosure herein without departing from the spirit or scope of the invention. The preceding description, therefore, is not meant to limit the scope of the invention. Rather, the scope of the invention is to be determined only by the appended claims and their equivalents.

1. A water-dispersible and substantially non-combustible liquid vitamin composition comprising:
a) from 10% to 60% by weight of a vitamin component selected from the group consisting of:
(i) one or more precursors of Vitamin A;
(ii) one or more precursors of Vitamin E;
(iii) a mixture of one or more precursors of Vitamin A and one or more precursors of Vitamin E;
(iv) a mixture of one or more precursors of Vitamin A and Vitamin D3;
(v) a mixture of one or more precursors of Vitamin E and Vitamin D3; and
(vi) a mixture of one or more precursors of Vitamin A, one or more precursors of Vitamin E, and Vitamin D3;
b) from 2% to 15% by weight of a C4 to C6 alkyl lactate;
c) from 20% to 50% by weight of one or more veterinarily acceptable emulsifiers;
d) from 1% to 15% by weight of water; and
e) from 2% to 10% of an oil;
wherein the flashpoint of the composition is about 200° F. or greater.

2. The composition of claim 1, wherein said composition further comprises from 1% to 15% by weight of a veterinarily acceptable stabilizer.

3. The composition of claim 2, wherein the stabilizer is propylene glycol.

4. The composition of claim 1, wherein the amount of the alkyl lactate is from 4% to 7%.

5. The composition of claim 1, wherein the composition is substantially free of a flammable alcohol.

6. The composition of claim 1, wherein the ratio of b) to d) is from 1:5 to 10:1.

7. The composition of claim 1, wherein the emulsifier is a non-ionic surfactant.

8. The composition of claim 1, further comprising from 0.5% to 6% by weight of an antioxidant.

9. The composition of claim 8, wherein the antioxidant is ethoxyquin, BHT or BHA.

10. The composition of claim 9, wherein the antioxidant is ethoxyquin.

11. The composition of claim 1, wherein the viscosity of the composition is from 1000 cP to 10000 cP at 0° C.

12. The composition of claim 1, wherein the viscosity of the composition is from 100 cP to 2000 cP at 15° C.

13. The composition of claim 1, wherein said composition adequately disperses into water within 2 minutes when added at a ratio of composition to water of from 1 g/kg to 50 g/kg.

14. The composition of claim 1, wherein the amount of water is from 1% to 5% by weight.

15. The composition of claim 1, wherein component a) is one or more precursors of Vitamin A.

16. The composition of claim 1, wherein component a) is one or more precursors of Vitamin E

17. The composition of claim 1, wherein component a) comprises a precursor of Vitamin A and Vitamin D3 and the weight/weight ratio of the precursor of Vitamin A to Vitamin D3 is from 100:1 to 40:1.

18. The composition of claim 15, wherein the total amount of the one or more precursors of Vitamin A in the composition is from 15 to 50% by weight.

19. The composition of claim 16, wherein the total amount of the one or more precursors of Vitamin E in the composition is from 10 to 60% by weight.

20. A water-dispersible and substantially non-combustible liquid vitamin composition comprising:
a) from 10% to 60% by weight of a vitamin component selected from the group consisting of:
(i) one or more precursors of Vitamin A;
(ii) one or more precursors of Vitamin E;
(iii) a mixture of one or more precursors of Vitamin A and one or more precursors of Vitamin E;
(iv) a mixture of one or more precursors of Vitamin A and Vitamin D3;
(v) a mixture of one or more precursors of Vitamin E and Vitamin D3; and
(vi) a mixture of one or more precursors of Vitamin A, one or more precursors of Vitamin E, and Vitamin D3;
b) from 3% to 15% by weight of a C1 to C3 alkyl lactate;
c) from 20% to 50% by weight of one or more veterinarily acceptable emulsifiers;
d) from 3% to 15% by weight of water; and
e) from 2% to 10% of an oil;
wherein the flashpoint of the composition is about 200° F. or greater.

21. The composition of claim 20, wherein said composition further comprises from 1% to 15% by weight of a veterinarily acceptable stabilizer.

22. The composition of claim 21, wherein the stabilizer is propylene glycol.

23. The composition of claim 24, wherein the amount of the C1-C3 alkyl lactate is from 3% to 12%.

24. The composition of claim 20, wherein the composition is substantially free of a mono-hydroxy alcohol.

25. The composition of claim 20, wherein the ratio of b) to d) is from 1:5 to 4:1.

26. The composition of claim 20, wherein the emulsifier is a non-ionic surfactant.

27. The composition of claim 20, further comprising from 0.5% to 6% by weight of an antioxidant.

29. The composition of claim 27, wherein the antioxidant is ethoxyquin, BHT or BHA.

30. The composition of claim 29, wherein the antioxidant is ethoxyquin.

31. The composition of claim 22, wherein the viscosity of the composition is from 1000 cP to 10000 cP at 0° C.

32. The composition of claim 20, wherein the viscosity of the composition is from 100 cP to 2000 cP at 15° C.

33. The composition of claim 20, wherein said composition adequately disperses into water within 2 minutes when added at a ratio of composition to water of from 1 g/kg to 50 g/kg.

34. The composition of claim 20, wherein the amount of water is from 3% to 6% by weight.

35. The composition of claim 20, wherein component a) is one or more precursors of Vitamin A.

36. The composition of claim 20, wherein component a) is one or more precursors of Vitamin E.

37. The composition of claim 20, wherein component a) comprises a precursor of Vitamin A and Vitamin D3 and the weight/weight ratio of the precursor of Vitamin A to Vitamin D3 is from 100:1 to 40:1.

38. The composition of claim 35, wherein the total amount of the one or more precursors of Vitamin A in the composition is from 15 to 50% by weight.

39. The composition of claim 36, wherein the total amount of the one or more precursors of Vitamin E in the composition is from 10% to 30% by weight.

40. The composition of claim 1, further comprising a fungicide.

41. The composition of claim 20, further comprising a fungicide.

42. A water-dispersible and substantially non-combustible liquid vitamin composition comprising:
a) from 1% to 6% by weight of Vitamin D3;
b) from 2% to 15% by weight of a C4 to C6 alkyl lactate;
c) from 20% to 50% by weight of one or more veterinarily acceptable emulsifiers;
d) from 1% to 15% by weight of water; and
e) from 5% to 30% by weight of an oil;
wherein the flashpoint of the composition is about 200° F. or greater.

43. A water-dispersible and substantially non-combustible liquid vitamin composition comprising:
a) from 1% to 6% by weight of Vitamin D3:
b) from 3% to 15% by weight of a C1 to C3 alkyl lactate;
c) from 20% to 50% by weight of one or more veterinarily acceptable emulsifiers;
d) from 1% to 15% by weight of water; and
e) from 5% to 30% by weight of an oil;
wherein the flashpoint of the composition is about 200° F. or greater.
